SEQUENCE LISTING (6 sequences)

SEQ ID NO: 1 - protein, 42 aa, Homo sapiens
  MOD_RES (42)..(42)
YAEGTFISDYSIAMDKIHQQDFVNWLLAQKGKKNDWKHNITQ

SEQ ID NO: 2 - protein, 30 aa, Homo sapiens
  MOD_RES (30)..(30): Lys is modified with an NH2 group
YAEGTFISDYSIAMDKIHQQDFVNWLLAQK

SEQ ID NO: 3 - protein, 31 aa, Artificial Sequence
  synthetic construct
  MOD_RES (2)..(2): Xaa is is Aminoisobutyric acid (Aib)
  MOD_RES (31)..(31): Xaa is Lys derivatized with (4,7,10-Tris- carboxymethyl-1,4,7,10-tetraaza-cyclododec-1-yl)-acetyl
  MOD_RES (31)..(31): Amidated C-terminus
YXEGTFISDLSIALDRIHQEEFIEWLLAGGX

SEQ ID NO: 4 - protein, 31 aa, Artificial Sequence; identical to SEQ ID NO: 3
  synthetic construct
  MOD_RES (2)..(2): Xaa is is Aminoisobutyric acid (Aib)
  MOD_RES (31)..(31): Xaa is Lys derivatized with (4,7,10-Tris- carboxymethyl-1,4,7,10-tetraaza-cyclododec-1-yl)-acetyl with complexed Gallium
  MOD_RES (31)..(31): Amidated C-terminus

SEQ ID NO: 5 - protein, 31 aa, Artificial Sequence
  synthetic construct
  MOD_RES (2)..(2): Xaa is is Aminoisobutyric acid (Aib)
  MOD_RES (31)..(31): Xaa is Cys derivatized with 2-[2-(4,7,10-Tris- carboxymethyl-1,4,7,10-tetraaza-cyclododec-1-yl)-ethanesulfonyl]- ethyl
  MOD_RES (31)..(31): Amidated C-terminus
YXEGTFISDLSIALDRIHQEEFIQWLLAGGX

SEQ ID NO: 6 - protein, 31 aa, Artificial Sequence; identical to SEQ ID NO: 5
  synthetic construct
  MOD_RES (2)..(2): Xaa is is Aminoisobutyric acid (Aib)
  MOD_RES (31)..(31): Xaa is Cys derivatized with 2-[2-(4,7,10-Tris- carboxymethyl-1,4,7,10-tetraaza-cyclododec-1-yl)-ethanesulfonyl]- ethyl with complexed Gallium
  MOD_RES (31)..(31): Amidated C-terminus

(19) United States
(12) Patent Application Publication
(10) Pub. No.: US 2023/0127047 A1
(43) Pub. Date: Apr. 27, 2023

(54) SELECTIVE GIP RECEPTOR AGONISTS COMPRISING A CHELATING MOIETY FOR IMAGING AND THERAPY PURPOSES

(71) Applicant: ANTAROS MEDICAL AB, Mölndal (SE)

(72) Inventors: Andreas Evers, Mölndal (SE); Michael Wagner, Mölndal (SE); Torsten Haack, Mölndal (SE); Martin Bossart, Mölndal (SE); Katrin Lorenz, Mölndal (SE)

(21) Appl. No.: 17/914,612

(22) PCT Filed: Mar. 30, 2021
(86) PCT No.: PCT/EP2021/058250

§ 371 Date: Sep. 26, 2022

(30) Foreign Application Priority Data
Mar. 31, 2020 (EP) 20315081.8

(51) Int. Cl. C07K 14/575; A61K 51/08; A61K 51/04

(52) U.S. Cl. CPC C07K 14/575; A61K 51/088; A61K 51/0474

(57) ABSTRACT

The present invention relates to GIP(1-30) analogues which selectively bind and activate the GIP receptor and comprise a chelating moiety capable of binding a metal ion and their use, for example in PET imaging or for radiotherapy.

### FIELD OF THE INVENTION

The present invention relates to GIP(1-30) analogs which selectively bind and activate the GIP receptor and comprise a chelating moiety capable of binding a metal ion. Preferred metal ions are radionuclides, e.g. detectable by positron emission tomography (PET) or single photon emission computed tomography (SPECT). The obtained compounds are useful for visualizing cells expressing the GIP receptor, in particular in the pancreas, as well as in a method of detecting and treating neuroendocrine tumors characterized by an overexpression of the GIP receptor.

### BACKGROUND OF THE INVENTION

GIP and GLP-1 are the two gut enteroendocrine cell-derived hormones accounting for the incretin effect, which accounts for over 70% of the insulin response to an oral glucose challenge (Baggio et al., Gastroenterology 2007, 132, 2131).

GIP (glucose-dependent insulinotropic polypeptide), also referred to as hGIP or hGIP(1-42), is a 42 amino acid peptide that is released from intestinal K-cells following food intake. GIP amino acid sequence is shown as SEQ ID NO: 1:

H₂N-YAEGTFISDYSIAMDKIHQQDFVNWLLAQKGKKNDWKHNITQ-OH

GIP and its analogs produce glucose-dependent insulin secretion from beta-cells thus exerting glucose control without risk for hypoglycemia. GIP exhibits glucoregulatory effects as a result of its direct effect on pancreatic islets (Taminato et al., Diabetes 1977, 26, 480; Adrian et al., Diabetologia 1978, 14, 413; Lupi et al., Regul Pept 2010, 165, 129). In addition, GIP analogs produce glucagon secretion from alpha cells in normal and diabetic humans (Chia et al., Diabetes 2009, 58, 1342; Christensen et al., Diabetes 2011, 60, 3103). This effect has the potential to further minimize hypoglycemic risk in diabetic subjects that lack hypoglycemia awareness. GIP peptides have also been shown to produce beneficial effect on bone and neuroprotection in preclinical models, effects if translated to humans may be of value in older diabetic subjects (Ding et al., J Bone Miner Res 2008, 23, 536; Verma et al., Expert Opin Ther Targets 2018, 22, 615; Christensen et al., J Clin Endocrinol Metab 2018, 103, 288). In addition, preclinical data indicates that GIP may have an anti-emetic effect and prevent emesis elicited by mechanisms (e.g. PYY) that induce nausea and vomiting in preclinical animal models (US 2018/0298070).

GIP(1-30) amide is a C-terminally truncated form of GIP, that is generated in vivo from the precursor proGIP in human intestine and pancreatic islets (Y. Fujita et al., Am J Physiol Gastrointest Liver Physiol 2010, 298, G608). It is described as fully bioactive and shows high agonistic activity at the hGIP receptor in a similar range as full-length native GIP (hGIP, GIP(1-42)) itself (M. Maletti et al., Diabetes 1987, 36, 1336; M. B. Wheeler et al., Endocrinology 1995, 136, 4629; L. Hansen et al., Br J Pharmacol. 2016, 173(5), 826). Volz et al, FEBS Lett. 1995 Oct. 2; 373(1):23-9, reported a K_{d} of 19.3 nM for GIP(1-42) and of 11.3 nM for GIP(1-30).

GIP(1-30) amide amino acid sequence is shown as SEQ ID NO: 2:

H₂N-YAEGTFISDYSIAMDKIHQQDFVNWLLAQK-NH₂

### PET

Positron emission tomography (PET) is a routinely used nuclear medicine imaging technique, capable of producing three dimensional images of subjects. After injection of a suitable radioactive tracer containing a positron-emitting radionuclide, a pair of orthogonal gamma rays is detected resulting from annihilation of a positron. Three dimensional images can be obtained after computational reconstruction; correct anatomical localization is frequently ensured by simultaneous recording of a CT X-ray scan.

Another nuclear medicine tomographic imaging technique to provide three dimensional images is single photon emission computed tomography (SPECT). This method is based on detection of gamma rays emitted by a suitable radioisotope.

These methods are generally utilized to examine tissues and to monitor physiological processes e.g. by using 18-fluorodeoxy glucose for monitoring of metabolic activity. Alternatively, a marker radioisotope can be attached to a specific ligand to create a radioligand displaying specificity to certain tissues or receptors, for example GPCR's.

Specific detection of a single receptor type by PET or SPECT requires a selective interaction of the tracer ligand with the receptor of interest.

An important prerequisite for a PET tracer is metabolic stability, since PET measures the total radioactivity concentrations in tissue and is not able to discern different radiolabeled chemical entities, such as radiolabeled metabolites. Peptidic PET tracers with improved metabolic stability are therefore preferred to enhance clearance of the intact PET tracer by the kidneys.

Selective GLP-1 receptor agonists with an imaging moiety have been described (WO2006024275; R. K. Selvaraju et al, Journal of Nuclear Medicine, 2013, 54, 1; O. Eriksson et al, J Clin Endocrinol Metab, 2014, 99(5), 1519).

The present invention comprises imaging ligands that selectively interact with the GIP receptor (GIPR).

Imaging ligands selective for the GIP receptor are of use for the visualization of neuroendocrine tumors (NET). Currently, somatostatin receptor targeting is considered the standard technique for visualization of neuroendocrine tumors. Not all NETs over-express somatostatin receptors, but many with low somatostatin receptor levels, express GIP receptor. GIP receptor targeting could offer a chance for visualizing these NETs. Therefore, there is a still a need for ligands strongly and selectively binding to the human GIP receptor.

Peptides derived from native GIP carrying a chelating moiety are described in the literature. Willekens et al. describe a N-acetylated GIP(1-42) analog with DTPA as chelating moiety (S. Willekens et al., Nature Sci Rep 2018, 8, 2948). While this analog shows selective binding to the GIP receptor, it leads to slow receptor internalization due to the N-acetylation (see Ismail et al. Mol. Cell. Endocrinol., 414, 202). Intact tracer and tracer metabolites labeled with a radiometal via the chelating moiety can be trapped in the lysosomes upon tracer internalization and degradation, resulting in enhanced tracer accumulation in the target cells. A high internalization rate is therefore desired in order to achieve the high target-to-background ratios required for high quality images.

Gourni et al describe GIP(1-30)amide analogs containing a DOTA chelating moiety which show similar to slightly lower binding affinity compared to native GIP(1-42). The tested compounds had IC50 values of between 1.5 and 2.5 nM and K_{d} values between 8.5 and 10.6 nM, representing binding affinities for the human GIP receptor about equal to GIP(1-30). (E. Gourni et al., J. Nucl. Med. 2014, 55, 976)

Peptide receptor radionuclide therapy (PRRT) is a molecular targeted therapy used to treat neuroendocrine tumors (NET). Molecular targeted therapies use drugs or other substances to identify and attack cancer cells while reducing harm to healthy tissue. PRRT delivers high doses of radiation to tumors in the body to destroy or slow their growth and reduce disease side effects. GIP receptor binders carrying a chelating moiety (e.g. DOTA) loaded with a suitable radionuclide e.g. (Y-90)³⁺, (In-111)³⁺ or (Lu-177)³⁺ have therefore high potential for the treatment of NETs with elevated expression of GIP receptors.

### BRIEF SUMMARY OF THE INVENTION

Provided herein are GIP(1-30) analogs which potently and selectively bind and activate the GIP receptor and comprise a chelating moiety capable of binding a metal ion, making the molecule suitable for imaging studies, for example PET studies.

The inventors have surprisingly found that peptides of the invention have a higher affinity towards the human GIP receptor compared to native GIP, selectively activate the GIP receptor (compared to the GLP-1 and the Glucagon receptor) and have suitable physicochemical properties, such as being soluble, and chemically as well as physically stable in aqueous solutions. Meanwhile, the potency of the peptides of the invention is similar to or lower than that of GIP(1-42) and GIP(1-30). For imaging purposes, it is preferable that the compounds possess a high target binding affinity, but since the agonistic effect is not sought in such applications, a low potency is preferred over a high potency. Thus, a compound showing a stronger binding affinity and at the same time a maintained or even reduced potency is very desirable e.g. for PET imaging.

Compounds of the present invention have been found to have a stability in plasma that allows the compounds to be used for imaging purposes.

The compounds of the present invention therefore are well suited for the investigation of the GIP receptor in vivo using imaging technologies, such as PET or SPECT.

Compared to native human GIP, the peptides of the invention are truncated at position 30, modified in 10 positions and have an additional amino acid at the C-terminus modified with a chelating moiety. These modifications surprisingly result in peptides with high chemical stability as well as higher affinity towards the GIP receptor with a substantially unchanged, potent agonistic activity.

The peptides of the present invention were found to exhibit a higher binding affinity (lower IC50 value) compared to known GIP(1-30) analogs.

The present invention therefore provides highly selective GIP receptor agonists which are well suited for the investigation of the GIP receptor in vivo using an imaging technology, for example the PET technology.

The invention provides a peptidic compound having the formula (I).

(I)
Tyr-Aib-Glu-Gly-Thr-Phe-Ile-Ser-Asp-Leu-Ser-Ile-
Ala-Leu-Asp-Arg-Ile-His-Gln-Glu-Glu-Phe-Ile-X24-
Trp-Leu-Leu-Ala-Gly-Gly-X31-R¹

wherein
X24 represents an amino acid selected from Glu or Gln,
X31 represents an amino acid selected from Cys(VS-DO3A), Cys(VS-NO2A), Cys(mal-DOTA), Cys(mal-NOTA), Cys(mal-NODAGA), Lys(DOTA), Lys(NOTA), Lys(PEG-DOTA) and Lys(VS-DO3A),
wherein DOTA, NOTA, DO3A, NO2A or NODAGA may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺,
R¹ represents OH or NH₂

or a salt or a solvate thereof.

A further embodiment of the invention provides a peptidic compound having the formula (I), wherein
X31 represents an amino acid Cys(VS-DO3A) or Lys(DOTA),
wherein DOTA or DO3A may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺.

A further embodiment of the invention provides a peptidic compound having the formula (I), wherein
X24 is Glu.

A further embodiment of the invention provides a peptidic compound having the formula (I), wherein
X24 is Glu, and
R¹ is OH.

A further embodiment of the invention provides a peptidic compound having the formula (I), wherein
X24 is Glu, and
R¹ is NH₂.

A further embodiment of the invention provides a peptidic compound having the formula (I), wherein
X24 is Gln.

A further embodiment of the invention provides a peptidic compound having the formula (I), wherein
X24 is Gln, and
R¹ is OH.

A further embodiment of the invention provides a peptidic compound having the formula (I), wherein
X24 is Gln, and
R¹ is NH₂.

A further embodiment of the invention provides a peptidic compound having the formula (I), wherein
X24 is Gln,
X31 is C(VS-DO3A).

A further embodiment of the invention provides a peptidic compound having the formula (I), wherein
X24 is Glu,
X31 is K(DOTA).
Specific examples of a peptidic compound of formula (I) are the compounds of
   SEQ ID NO: 3 and 4, 5 and 6 as well as salts or solvates thereof.

A further embodiment of the invention provides a peptidic compound having the formula (I), wherein
DOTA or DO3A is unloaded.

A further embodiment of the invention provides a peptidic compound having the formula (I) wherein
DOTA or DO3A is loaded with a metal ion selected from Gd³⁺, Ga³⁺, Cu²⁺, (Al—F)²⁺, Y³⁺, Tc³⁺, In³⁺, Lu³⁺ and Re³⁺.

A further embodiment of the invention provides a peptidic compound having the formula (I) wherein
DOTA or DO3A is loaded with a metal ion Ga³⁺.

A further embodiment of the invention provides a peptidic compound having the formula (I) wherein
DOTA or DO3A is loaded with a metal ion Gd³⁺.

A further embodiment of the invention provides a peptidic compound having the formula (I) wherein
DOTA or DO3A is loaded with a metal radionucleotide ion (Cu-64)²⁺, (Ga-68)³⁺, (Al—F-18)²⁺, (Y-86)³⁺.

A further embodiment of the invention provides a peptidic compound having the formula (I) wherein
DOTA or DO3A is loaded with one metal radionucleotide ion (Ga-67)³⁺, (Tc-99)³⁺, (In-111)³⁺.

A further embodiment of the invention provides a peptidic compound having the formula (I) wherein
DOTA or DO3A is loaded with one metal radionucleotide ion selected from (Cu-67)²⁺, (Y-90)³⁺, (In-111)³⁺, (Lu-177)³⁺, (Re-186)³⁺ and (Re-188)³⁺.

Preferred compounds are the peptides with SEQ ID No. 3 to 6 listed in table 1 or a salt or a solvate thereof.

TABLE 1
Sequences

| SEQ. ID | Sequence |
|---|---|
| 1 | Y-A-E-G-T-F-I-S-D-Y-S-I-A-M-D-K-I-H-Q-Q- |
| | D-F-V-N-W-L-L-A-Q-K-G-K-K-N-D-W-K-H-N-I- |
| | T-Q-OH |
| 2 | Y-A-E-G-T-F-I-S-D-Y-S-I-A-M-D-K-I-H-Q-Q- |
| | D-F-V-N-W-L-L-A-Q-K-NH2 |
| 3 | Y-Aib-E-G-T-F-I-S-D-L-S-I-A-L-D-R-I-H-Q- |
| | E-E-F-I-E-W-L-L-A-G-G-K(DOTA)-NH2 |
| 4 | Y-Aib-E-G-T-F-I-S-D-L-S-I-A-L-D-R-I-H-Q- |
| | E-E-F-I-E-W-L-L-A-G-G-K(DOTA(Ga))-NH2 |
| 5 | Y-Aib-E-G-T-F-I-S-D-L-S-I-A-L-D-R-I-H-Q- |
| | E-E-F-I-Q-W-L-L-A-G-G-C(VS-DO3A)-NH2 |
| 6 | Y-Aib-E-G-T-F-I-S-D-L-S-I-A-L-D-R-I-H-Q- |
| | E-E-F-I-Q-W-L-L-A-G-G-C(VS-DO3A(Ga))-NH2 |

The compounds of the invention are capable of specifically binding to the GIP receptor. The compounds of the invention are GIP receptor agonists as determined by the observation that they are capable of stimulating intracellular cAMP formation upon binding at the receptor for GIP. The compounds exhibit at least a relative activity of 0.1%, preferably 0.5%, more preferably 1.0% and even more preferably 10.0% compared to that of natural GIP at the GIP receptor.

The compounds of the invention do not activate the GLP-1 receptor and the Glucagon receptor significantly as determined by the observation that they are not capable of stimulating intracellular cAMP formation upon binding at the receptor for GLP-1 or Glucagon, respectively, at the chosen concentrations. The EC50 of a given compound of this invention at the GLP-1 receptor or Glucagon receptor is higher than 100 pM, more preferably higher than 1000 pM and even more preferably higher than 10000 pM in the respective assay system as described in Example 5.

The inventors of the present invention found that peptidic compounds of the formula I with Aib at position 2, Leu at position 10 and 14, Arg at position 16, Glu at position 20 and 21, Ile at position 23, Glu or Gln at position 24, Gly at position 29 and 30, and the chelating unit attached to a C-terminal amino acid (Lys or Cys) at position 31 show high affinity towards the human GIP receptor as determined via the method used for Example 6.

Further, the compounds of the invention preferably have a favourable chemical stability at physiological pH values, e.g., at pH 7.0, pH 7.3 or pH 7.4 at 4° C., 25° C. or 40° C. Preferably, the purity of the compounds in these buffers after 7 days at 40° C. is greater than 90%.

In addition, compounds of the invention carry an Aib at position 2 to stabilize against DPP-IV cleavage. Native GIP has been shown to be a substrate of DPP-IV, leading to a cleavage between Ala at position 2 and Glu at position 3 (see Mentlein et al. Eur. J. Biochem. 1993, 214, 829). The incorporation of an Aib at position 2 is inhibiting the DPP-IV cleavage and therefore contributes to an increased metabolic stability.

Furthermore, the compounds of the invention contain a chelating moiety capable of binding a metal ion, making the molecule suitable for imaging studies, for example PET or SPECT studies. The chelating moiety represents a non-cyclic or cyclic structure containing electron pair donating elements to ensure strong binding to the metal cation. Strong chelation is a prerequisite for use as an imaging modality in order to prevent leaching of the radioisotope, which may result in systemic toxicity, increased background signal and reduction of the signal at the area of interest. Choice of an optimal chelating moiety depends on the nature of the complexed radiometal. Exemplifying frequently used chelating moieties and their corresponding names are listed in Scheme 1, more examples can e.g. be found in T. J. Wadas et al, Chem. Rev. 2010, 110, 2858.

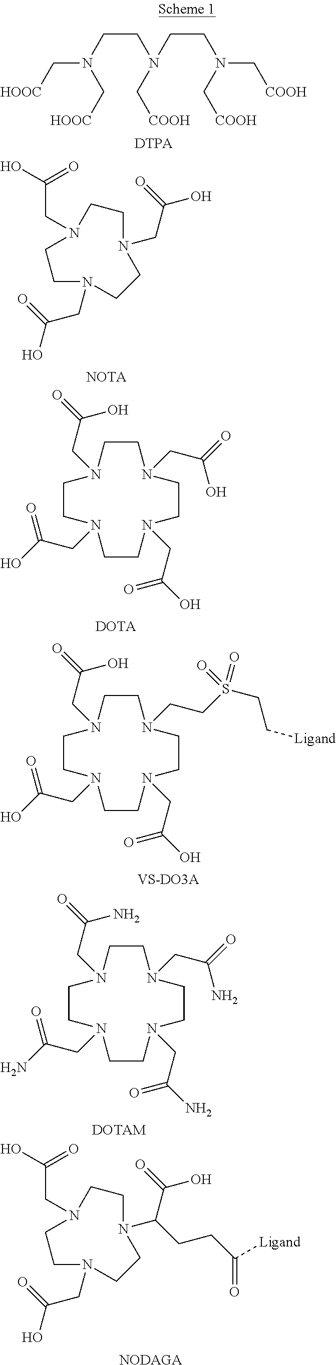

In certain embodiments, i.e. when the compound of formula (I) comprises genetically encoded amino acid residues, the invention further provides a nucleic acid (which may be DNA or RNA) encoding said compound, an expression vector comprising such a nucleic acid, and a host cell containing such a nucleic acid or expression vector.

In a further aspect, the present invention provides a composition comprising a compound of the invention in admixture with a carrier. In preferred embodiments, the composition is a pharmaceutically acceptable composition and the carrier is a pharmaceutically acceptable carrier. The compound of the invention may be in the form of a metal complex, e.g. a gallium(III) complex, a salt, e.g. a pharmaceutically acceptable salt, or a solvate, e.g. a hydrate. In still a further aspect, the present invention provides a composition for use in a method of medical treatment including diagnostic treatment, particularly in human medicine.

Compounds of this invention and formulation thereof may primarily be used to visualize the GIP receptor in living subjects and relevant tissues, preferably using the PET technology.

It is noted that the invention relates to all possible combinations of features recited in the claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

### DETAILED DESCRIPTION OF THE INVENTION

### Definitions

The amino acid sequences of the present invention contain the conventional one letter and three letter codes for naturally occurring amino acids, as well as generally accepted three letter codes for other amino acids, such as Aib (alpha-amino-isobutyric acid), or Nle (Norleucine).

The invention provides peptidic compounds as defined above.

The peptidic compounds of the present invention comprise a linear backbone of amino carboxylic acids linked by peptide, i.e. carboxamide bonds. Preferably, the amino carboxylic acids are α-amino carboxylic acids and more preferably L-α-amino carboxylic acids, unless indicated otherwise. The peptidic compounds comprise a backbone sequence of 31 amino carboxylic acids.

For the avoidance of doubt, in the definitions provided herein, it is generally intended that the sequence of the peptidic moiety differs from native GIP at least at one of those positions which are stated to allow variation. Amino acids within the peptide moiety can be considered to be numbered consecutively from 1 to 42 in the conventional N-terminal to C-terminal direction. Reference to a “position” within peptidic moiety should be constructed accordingly, as should reference to positions within native exendin-4 and other molecules, e.g., in GIP, Tyr is at position 1, Ala at position 2, . . . , Met at position 14, . . . and Gln at position 42.

In a further aspect, the present invention provides a composition comprising a compound of the invention as described herein, a metal complex, or a salt or solvate thereof, in admixture with a carrier.

The invention also provides a composition wherein the composition is a pharmaceutically acceptable composition, and the carrier is a pharmaceutically acceptable carrier.

### Peptide Synthesis

The skilled person is aware of a variety of different methods to prepare peptides that are described in this invention. These methods include but are not limited to synthetic approaches and recombinant gene expression. Thus, one way of preparing these peptides is the synthesis in solution or on a solid support and subsequent isolation and purification. A different way of preparing the peptides is gene expression in a host cell in which a DNA sequence encoding the peptide has been introduced. Alternatively, the gene expression can be achieved without utilizing a cell system. The methods described above may also be combined in any way.

A preferred way to prepare the peptides of the present invention is solid phase synthesis on a suitable resin. Solid phase peptide synthesis is a well established methodology (see for example: Stewart and Young, Solid Phase Peptide Synthesis, Pierce Chemical Co., Rockford, III., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g. a trityl resin, a chlorotrityl resin, a Wang resin or a Rink amide resin in which the linkage of the carboxy group (or carboxamide for Rink resin) to the resin is sensitive to acid (when Fmoc strategy is used). The polymer support must be stable under the conditions used to deprotect the α-amino group during the peptide synthesis.

After the first amino acid has been coupled to the solid support, the α-amino protecting group of this amino acid is removed. The remaining protected amino acids are then coupled one after the other in the order represented by the peptide sequence using appropriate amide coupling reagents, for example BOP, HBTU, HATU or DIC (N,N′-diisopropylcarbodiimide)/HOBt (1-hydroxybenzotriazol), wherein BOP, HBTU and HATU are used with tertiary amine bases. Alternatively, the liberated N-terminus can be functionalized with groups other than amino acids, for example carboxylic acids, etc.

Finally the peptide is cleaved from the resin and deprotected. This can be achieved by using King's cocktail (D. S. King, C. G. Fields, G. B. Fields, Int. J. Peptide Protein Res. 36, 1990, 255-266). The raw material can then be purified by chromatography, e.g. preparative RP-HPLC, if necessary.

The synthesized peptide is then further modified by attaching a side chain which contains a chelating moiety capable of integration a metal ion, for example Ga³⁺. In those cases where the attachment point in the peptide backbone is a lysine, the side chain can be linked by the reaction with a suitable amidation group, e.g. a hydroxyl succinimide ester, or with a vinylsulfone group via Michael addition. In other cases, when the attachment side is a thiol of a cysteine, the side chain can be connected by the reaction with a maleimide functionality or with a vinylsulfone group via Michael addition. The raw material can then be deprotected as necessary and purified by chromatography, e.g. preparative RP-HPLC. The side chains attached to the compounds of this invention are summarized in table 2.

For the compounds of the present invention the building blocks listed in table 3 may be used. With the exception of the building blocks VS-DO3A and VS-NO2A these building blocks are commercially available. The synthesis of VS-DO3A is described in Example 1, the synthesis of VS-NO2A can be performed in an analogous way.

TABLE 2
Side chains

| Side chain | Side chain structure | Side chain name |
|---|---|---|
| mal-DOTA | 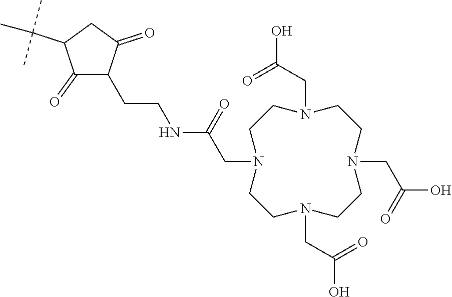 | 2,5-dioxo-1-{2-[2- (4,7,10-tris- carboxymethyl- 1,4,7,10tetraaza- cyclododec-1-yl)- acetylamino]-ethyl}- pyrrolidin-3-yl |
| mal-NOTA | 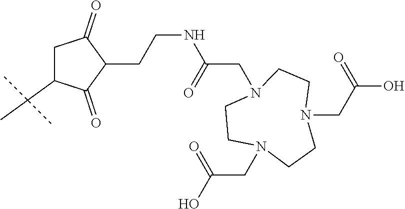 | 1-{2-[2-(4,7-bis- carboxymethyl- [1,4,7]triazonan-1-yl)- acetylamino]-ethyl}-2,5- dioxo-pyrrolidin-3-yl |
| mal- NODAGA | 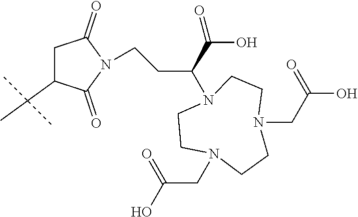 | 1-{2-[(S)-4-(4,7-bis- carboxymethyl- [1,4,7]triazonan-1-yl)-4- carboxy-butyrylamino]- ethyl}-2,5-dioxo- pyrrolidin-3-yl |
| DOTA | 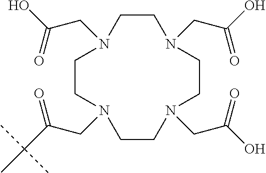 | (4,7,10-Tris- carboxymethyl-1,4,7,10- tetraaza-cyclododec-1- yl)-acetyl |
| NOTA | 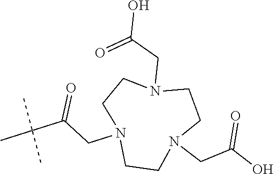 | (4,7-Bis-carboxymethyl- [1,4,7]triazonan-1-yl)- acetyl |
| PEG- NOTA | 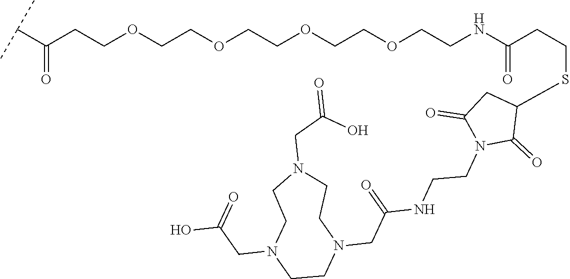 | 3-{2-[2-(2-{2-[3-(1-{2-[2- (4,7-Bis-carboxymethyl- [1,4,7]triazonan-1-yl)- acetylamino]-ethyl}-2,5- dioxo-pyrrolidin-3- ylsulfanyl)-propionyl amino]-ethoxy}-ethoxy)- ethoxy]-ethoxy}- propionyl |
| VS-NO2A | 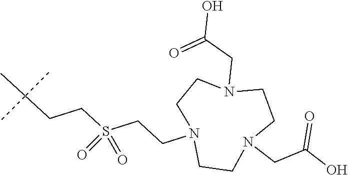 | 2-[2-(4,7-bis-carboxy methyl-[1,4,7]triazonan- 1-yl)-ethanesulfonyl]- ethyl |
| VS-DO3A | 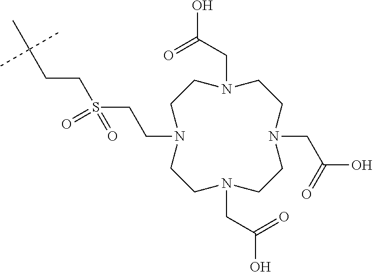 | 2-[2-(4,7,10-tris-carboxy methyl- 1,4,7,10tetraaza- cyclododec-1-yl)-ethane sulfonyl]-ethyl |

TABLE 3
Side chain building blocks

| Side chain building block | Building block structure |
|---|---|
| mal-DOTA building block | 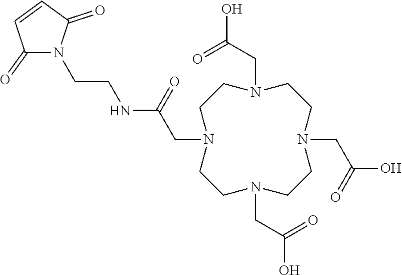 [SMILES: O=C(O)CN1CCN(CC(=O)O)CCN(CC(=O)NCCN2C(=O)C=CC2=O)CCN(CC(=O)O)CC1] |
| mal-NOTA building block | 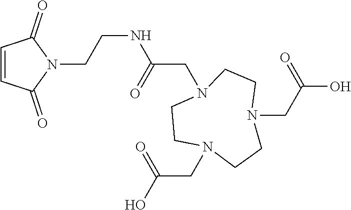 [SMILES: O=C(O)CN1CCN(CC(=O)O)CCN(CC(=O)NCCN2C(=O)C=CC2=O)CC1] |
| mal-NODAGA building block | 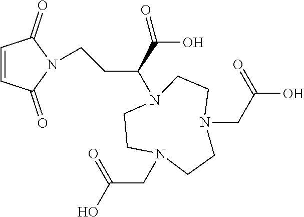 [SMILES: O=C(O)CN1CCN(CC(=O)O)CCN([C@@H](CCN2C(=O)C=CC2=O)C(=O)O)CC1] |
| DOTA building block | 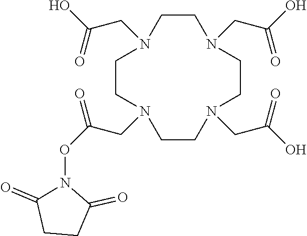 [SMILES: O=C(O)CN1CCN(CC(=O)O)CCN(CC(=O)ON2C(=O)CCC2=O)CCN(CC(=O)O)CC1] |
| NOTA building block | 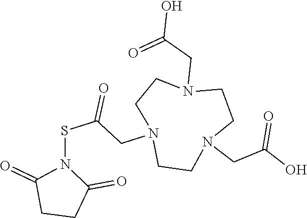 [SMILES: O=C(O)CN1CCN(CC(=O)O)CCN(CC(=O)SN2C(=O)CCC2=O)CC1] |
| SPDP-dPEG4-NHS ester | 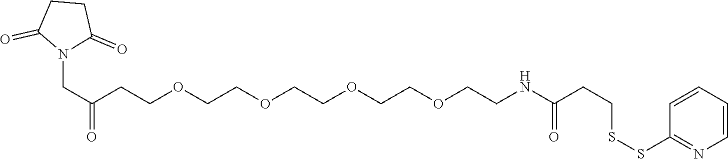 [SMILES: O=C(CCOCCOCCOCCOCCNC(=O)CCSSc1ccccn1)CN1C(=O)CCC1=O] |
| VS-NO2A building block | 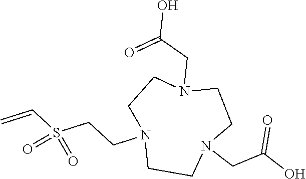 [SMILES: C=CS(=O)(=O)CCN1CCN(CC(=O)O)CCN(CC(=O)O)CC1] |
| VS-DO3A building block | 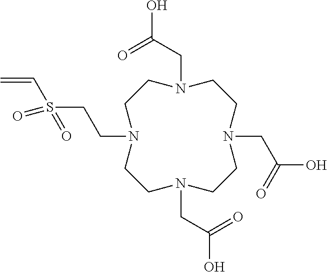 [SMILES: C=CS(=O)(=O)CCN1CCN(CC(=O)O)CCN(CC(=O)O)CCN(CC(=O)O)CC1] |

The complexing moiety at the side chain of the peptide can further be charged with a suitable metal ion, e.g. Ga³⁺. To achieve this, the peptide with the side chain is heated with a suitable salt of the desired cation in a suitable solvent. The raw material can then be purified by chromatography, e.g. preparative RP-HPLC or SPE, if necessary.

### Potency

As used herein, the term “potency” or “in vitro potency” is a measure for the ability of a compound to activate the receptors for GIP, GLP-1 or Glucagon in a cell-based assay. Numerically, it is expressed as the “EC50 value”, which is the effective concentration of a compound that induces a half maximal increase of response (e.g. formation of intracellular cAMP) in a dose-response experiment.

### Binding

The term “binding” as used herein preferably refers to the capability of a compound to bind to the human GIP receptor. Sometimes, reference may also be made to the term “affinity” instead of “binding”. More preferably the term “binding” as used herein refers to the capability of a compound to displace a radioactively labelled compound from the respective receptor in the binding assay, e.g. [¹²⁵I]GIP from the GIP receptor as described in Methods and shown in Examples. Numerically, it is expressed as the “IC50 value”, which is the effective concentration of a compound that displaces half of the radioactively labelled compound from the receptor in a dose-response experiment.

The compounds of the invention preferably have an IC50 for hGIP receptor of 10 nM or less, preferably of 8 nM or less, more preferably of 5 nM or less, more preferably of 3.13 nM or less, and even more preferably of 1 nM or less. The IC50 for the hGIP receptor may be determined as described in the Methods herein and as used to generate the results described in Example 6.

### Therapeutic Uses & Diagnostic Uses

The term diagnostic use refers to a use for detection and/or quantification of GIP receptors in living subjects and relevant tissues.

This includes but is not limited to determination of a receptor occupancy state of a given dose of a therapeutic binding to the GIP receptor in specific tissues. The receptor for GIP is broadly expressed in peripheral tissues including pancreatic islets, adipose tissue, stomach, small intestine, heart, bone, lung, kidney, testis, adrenal cortex, pituitary, endothelial cells, trachea, spleen, thymus, thyroid and brain. Consistent with its biological function as incretin hormone, the pancreatic ß-cells express the highest levels of the receptor for GIP in humans. Receptor occupancy studies represent an option to identify an optimal dose of therapeutic agents influencing the GIP receptor, including e.g. Selective GIP receptor agonists, e.g. compounds disclosed in WO 2012/055770, WO 2018/181864, WO 2019/211451, and WO 2016/066744; Dual GLP-1/GIP agonists, e.g. RG-7685 (MAR-701), RG-7697 (MAR-709, NN9709), BHM081, BHM089, BHM098, LBT-6030, ZP-I-70), TAK-094, SAR438335, Tirzepatide (LY3298176) or compounds disclosed in WO2014/096145, WO2014/096148, WO2014/096149, WO2014/096150 and WO2020/023386; or Triple GLP-1/glucagon/GIP receptor agonists (e.g. Tri-agonist 1706 (NN9423), HM15211). Furthermore, a GIP receptor scintigraphy is particularly applicable in the diagnosis of diseases characterized by increased presence of cells strongly expressing the GIP receptor, e.g. gastric, duodenal, ileal, pancreatic, and bronchial neuroendocrine tumors, as well as insulinomas and medullary thyroid carcinoma.

A person skilled in the art will be able to select a suitable metal ion for loading depending on the intended imaging technology. This includes, but is not limited to (Gd-68)³⁺ for MRI, (Cu-64)²⁺, (Ga-68)³⁺, (Al—F-18)²⁺ or (Y-86)³⁺ for PET or (Ga-67)³⁺, (Tc-99m)³⁺ or (In-111)³⁺ for SPECT measurements.

The term “therapeutic use” indicates an application of peptides described in the current invention for use in radiotherapy. This involves loading of said peptide with a suitable radionuclide like (Cu-67)²⁺, (Y-90)³⁺, (In-111)³⁺, (Lu-177)³⁺, (Re-186)³⁺ or (Re-188)³⁺, purification and quality control.

A preparation is considered suitable, if a loading efficacy of >98% can be obtained. The radioactive preparation can be injected as a part of an acceptable pharmaceutical composition into a patient. The applied dose is selected by a physician depending on considerations on e.g. the intended use (therapeutic or diagnostic), disease state (benign ort malignant), tumor size and location and loaded radioisotope.

### Pharmaceutical Compositions

The term “pharmaceutical composition” indicates a mixture containing ingredients that are compatible when mixed and which may be administered. A pharmaceutical composition may include one or more bioactive molecules. Additionally, the pharmaceutical composition may include carriers, solvents, adjuvants, emollients, expanders, stabilizers and other components, whether these are considered active or inactive ingredients. Guidance for the one skilled in preparing pharmaceutical compositions may be found, for example, in Remington: The Science and Practice of Pharmacy, (20th ed.) ed. A. R. Gennaro A. R., 2000, Lippencott Williams & Wilkins.

The GIP derivatives of the present invention or metal complexes or salts or solvates thereof, are administered in conjunction with an acceptable pharmaceutical carrier, diluent, or excipient as part of a pharmaceutical composition. A “pharmaceutically acceptable carrier” is a carrier which is physiologically acceptable while retaining the therapeutic properties of the substance with which it is administered. Standard acceptable pharmaceutical carriers and their formulations are known to one skilled in the art and described, for example, in Remington: The Science and Practice of Pharmacy, (20th ed.) ed. A. R. Gennaro A. R., 2000, Lippencott Williams & Wilkins. One exemplary pharmaceutically acceptable carrier is physiological saline solution.

Acceptable pharmaceutical carriers or diluents include those used in formulations suitable for oral, rectal, nasal or parenteral (including subcutaneous, intramuscular, intravenous, intradermal, and transdermal) administration. The compounds of the present invention will typically be administered intravenously.

The term “salt” or “pharmaceutically acceptable salt” means salts of the compounds of the invention which are safe and effective for use in mammals. Pharmaceutically acceptable salts may include, but are not limited to, acid addition salts and basic salts. Examples of acid addition salts include chloride, sulfate, hydrogen sulfate, (hydrogen) phosphate, acetate, trifluoroacetate, citrate, tosylate or mesylate salts. Examples of basic salts include salts with inorganic cations, e.g. alkaline or alkaline earth metal salts such as sodium, potassium, magnesium or calcium salts and salts with organic cations such as amine salts. Further examples of pharmaceutically acceptable salts are described in Remington: The Science and Practice of Pharmacy, (20th ed.) ed. A. R. Gennaro A. R., 2000, Lippencott Williams & Wilkins or in Handbook of Pharmaceutical Salts, Properties, Selection and Use, e.d. P. H. Stahl, C. G. Wermuth, 2002, jointly published by Verlag Helvetica Chimica Acta, Zurich, Switzerland, and Wiley-VCH, Weinheim, Germany.

The term “solvate” means complexes of the compounds of the invention or salts thereof with solvent molecules, e.g. organic solvent molecules and/or water.

The term “metal complex” means a chelate complex of the compounds of the invention with metal ions (e.g. of transition metals) wherein a polydentate (multiple bonded) ligand is a part of the compound that bonds to the metal ion through several of the ligand's atoms; (ligands with 2, 3 or 4 bonds to the metal ion are common).

Pharmaceutical compositions of the invention are those suitable for parenteral (for example subcutaneous, intramuscular, intradermal or intravenous), oral, rectal, topical and peroral (for example sublingual) administration, although the most suitable mode of administration depends in each individual case on the specific use of the bioactive ingredient and on the nature of the compound of formula (I) used in each case. Typically the route of administration for the intended use for the compounds of this invention is intravenous administration.

### Methods

Abbreviations employed are as follows:
AA amino acid
ACN acetonitrile
Aib alpha-amino-isobutyric acid, 2-methylalanine
cAMP cyclic adenosine monophosphate
Boc tert-butyloxycarbonyl
BSA bovine serum albumin
BOP (benzotriazol-1-yloxy)tris(dimethylamino)phosphonium hexafluorophosphate
tBu tertiary butyl
CT computer tomography
CTC 2-chlorotrityl chloride
DCM dichloromethane
DIC N,N′-diisopropylcarbodiimide
DIPEA N,N-diisopropylethylamine
DMEM Dulbecco's modified Eagle's medium
DMF dimethyl formamide
EDT ethanedithiol
FA formic acid
FBS fetal bovine serum
Fmoc fluorenylmethyloxycarbonyl
GCG Glucagon
GIP glucose-dependent insulinotropic polypeptide
GIPR GIP receptor
GLP-1 glucagon-like peptide 1
GLP-1R GLP-1 receptor
HATU 2-(1H-7-azabenzotriazol-1-yl)-1,1,3,3-tetramethyl uronium hexafluorophosphate
HBSS Hanks' Balanced Salt Solution
HBTU 2-(1H-benzotriazol-1-yl)-1,1,3,3-tetramethyl-uronium hexafluorophosphate
HOBt 1-hydroxybenzotriazole
HPLC High Performance Liquid Chromatography
LC/MS Liquid Chromatography/Mass Spectrometry
M molar
MBHA 4-methylbenzhydrylamine
ml milliliter
mM millimolar
mmol millimole(s)
μM micromolar
pmol micromole(s)
n.a. not available
n.d. not determined
nM nanomolar
nmol nanomole(s)
NMP N-methyl pyrrolidone
MRI magnetic resonance imaging
Pbf 2,2,4,6,7-pentamethyldihydro-benzofuran-5-sulfonyl
PEG polyethylene glycole
PET positron emission tomography
pM picomolar
PRRT peptide receptor radionuclide therapy
RP-HPLC reversed-phase high performance liquid chromatography
s.c. subcutaneous
SPE solid phase extraction
SPECT single photon emission computed tomography
TFA trifluoroacetic acid
TFE trifluorethanol
TRIS tris(hydroxymethyl)-aminomethan
Trt trityl
UHPLC Ultra High Performance Liquid Chromatography
UV ultraviolet

### General Synthesis of Peptidic Compounds

### Materials:

For solid phase peptide synthesis Rink-Amide resin (4-(2′,4′-Dimethoxyphenyl-Fmoc-aminomethyl)-phenoxyacetamido-norleucylaminomethyl resin) was used. Rink-Amide resin was purchased from Novabiochem with a loading of 0.35-0.43 mmol/g.

Fmoc protected natural and special amino acids were purchased from Protein Technologies Inc., Senn Chemicals, Merck Biosciences, Novabiochem, Iris Biotech or Bachem. The following standard amino acids were used throughout the syntheses: Fmoc-L-Ala-OH, Fmoc-L-Arg(Pbf)-OH, Fmoc-L-Asn(Trt)-OH, Fmoc-L-Asp(OtBu)-OH, Fmoc-L-Gln(Trt)-OH, Fmoc-L-Glu(OtBu)-OH, Fmoc-Gly-OH, Fmoc-L-His(Trt)-OH, Fmoc-L-Ile-OH, Fmoc-L-Leu-OH, Fmoc-L-Lys(Boc)-OH, Fmoc-L-Phe-OH, Fmoc-L-Pro-OH, Fmoc-L-Ser(tBu)-OH, Fmoc-L-Thr(tBu)-OH, Fmoc-L-Trp(Boc)-OH, Fmoc-L-Tyr(tBu)-OH, Fmoc-L-Val-OH, Fmoc-L-Cys(Trt)-OH, Fmoc-Aib-OH.

The solid phase peptide syntheses were performed on a Prelude Peptide Synthesizer (Protein Technologies Inc) using standard Fmoc chemistry and HBTU/DIPEA activation. DMF was used as the solvent. Deprotection: 20% piperidine/DMF for 2×2.5 min. Washes: 7×DMF. Coupling: 2:5:10 200 mM AA/500 mM HBTU/2M DIPEA in DMF 2× for 20 min. Washes: 5×DMF. Final wash after the last deprotection: 9×DCM, resin dried with N₂.

All the peptides that had been synthesized were cleaved from the resin with King's cleavage cocktail consisting of 82.5% TFA, 5% phenol, 5% water, 5% thioanisole, 2.5% EDT. The crude peptides were then precipitated in diethyl or diisopropyl ether, centrifuged, and lyophilized. Peptides were analyzed by analytical HPLC and checked by ESI mass spectrometry (see Table 4). Crude peptides were purified by a conventional preparative RP-HPLC purification procedure.

### General Preparative HPLC Purification Procedure:

The crude peptides were purified either on an Äkta Purifier System, a Jasco semiprep HPLC System, an Agilent 1100 HPLC system or a similar HPLC system. Preparative RP-C18-HPLC columns of different sizes and with different flow rates were used depending on the amount of crude peptide to be purified, e.g. the following columns have been used: Waters XSelect CSH C18 OBD Prep 5 μm 30×250 mm, Waters SunFire C18 OBD Prep 5 μm 30×250 mm, and Waters SunFire C18 OBD Prep 5 μm 50×150 mm. Acetonitrile (B) and water+0.1% TFA (A) or Acetonitrile (B) and water+0.1% FA (A) were employed as eluents. Product-containing fractions were collected and lyophilized to obtain the purified product, typically as TFA salt.

### Analytical HPLC/UHPLC

### Method A: Detection at 214 nm

column: Waters ACQUITY UPLC® CSH™ C18 1.7 μm (150×2.1 mm) at 50° C. solvent: H₂O+0.05% TFA: ACN+0.045% TFA (flow 0.5 ml/min)
gradient: 80:20 (0 min) to 80:20 (3 min) to 25:75 (23 min) to 5:95 (23.5 min) to 5:95 (26.5 min) to 80:20 (27 min) to 80:20 (33 min)
optionally with mass analyzer: LCT Premier, electrospray positive ion mode

### Method B: Detection at 214 nm

column: Waters ACQUITY UPLC® CSH™ C18 1.7 μm (150×2.1 mm) at 50° C. solvent: H₂O+0.05% TFA:ACN+0.035% TFA (flow 0.5 ml/min)
gradient: 80:20 (0 min) to 80:20 (3 min) to 25:75 (23 min) to 2:98 (23.5 min) to 2:98 (30.5 min) to 80:20 (31 min) to 80:20 (37 min)
mass analyzer: Agilent 6230 Accurate-Mass TOF or Agilent 6550 iFunnel Q-TOF; both equipped with a Dual Agilent Jet Stream ESI ion source.

### Method C: Detection at 214 nm

column: Waters ACQUITY UPLC® CSH™ C18 1.7 μm (150×2.1 mm) at 70° C. solvent: H₂O+0.05% TFA:ACN+0.035% TFA (flow 0.5 ml/min)
gradient: 63:37 (0 min) to 63:37 (3 min) to 45:55 (23 min) to 2:98 (23.5 min) to 2:98 (30.5 min) to 63:37 (31 min) to 63:37 (38 min)
mass analyzer: Agilent 6230 Accurate-Mass TOF, Agilent Jet Stream ESI

### Method D: Detection at 215 and 280 nm

column: Waters ACQUITY UPLC® BEH130 C18 1.7 μm column (2.1×100 mm) at 40° C.
solvent: H₂O+0.1% FA:ACN+0.1% FA (flow 0.5 ml/min)
gradient: 90:10 (0 min) to 10:90 (19.2 min) to 10:90 (20 min)

### Solubility Assessment

For solubility testing the compounds were added to an aqueous buffer at a target concentration of 0.5 mg/mL and were shaken for several minutes. Depending on the presence of remaining material as judged by visual inspection the compounds were determined to be soluble at the target concentration in the respective buffer system.

Solubility buffer system A) 100 mM Phosphate buffer pH 7.4

Solubility buffer system B) Acetate buffer pH 4.5

Solubility buffer system C) 100 mM Histidine buffer pH 5.5

Solubility buffer system D) 10 mM Glycine pH 2.5

Solubility buffer system E) 10 mM Glycine pH 2.0

Solubility buffer system F) 50 mM TRIS buffer, 30 mM m-cresol, 85 mM sodium chloride, 8 μM polysorbate 20 pH 7.3

### Chemical Stability Testing of Peptides

For chemical stability testing, the target concentration was 0.5 mg/mL pure compound in a pH 7.3 TRIS buffer (50 mM) containing m-cresol (30 mM), sodium chloride (85 mM) and polysorbate 20 (8 μM). The solution was stored for 7 days at 4° C., or 40° C. After that time, the solution was analysed by UHPLC (Analytical UHPLC Method D).

The “% Purity” after 7 days is defined by the % Relative purity at day 7 in relation to the % Relative purity at t0 following the equation

% Purity=[(% Relative purity t7)×100)]/% Relative purity t0

The % Relative purity at t0 was calculated by dividing the peak area of the peptide at t0 by the sum of all peak areas at t0 following the equation

% Relative purity t0=[(peak area t0)×100]/sum of all peak areas t0

Likewise, the % relative purity t7 was calculated by dividing the peak are of the peptide at t7 by the sum of all peak areas at t7 following the equation

% Relative purity t7=[(peak area t7)×100]/sum of all peak areas t7

### Plasma Stability Testing of Peptides

Blood plasma from a cynomolgus monkey (0.5 mL) was incubated with [⁶⁸Ga]Ga-S02-GIP-T4 (SEQ ID NO: 4) (3-5 MBq) for 0, 10, 45, and 90 min at 37° C. Then, 0.5 mL of acetonitrile was added to precipitate proteins, and the vials (Eppendorf 5415R centrifuge, Eppendorf AG, Hamburg, Germany) were centrifuged at 13200 rpm for 1 min at 4° C. The supernatant was transferred into 0.2 μm nylon membrane filter (Corning Incorporated, Corning, N.Y., USA) that was centrifuged at 13200 rpm and 4° C. for 1 min. The radioactivity of the supernatant, pellet, and filter was measured in well-type in-house built NaI(Tl) scintillation counter and corrected for dead-time and decay. The data was used to calculate the recovery of the sample (>95%). The supernatant (0.5 mL) was diluted with 1.5 mL of deionized water and spiked with 10 μL of standard reference (S02-GIP-T4 solution of 1 mg/mL concentration) and analyzed (1.8 mL) on UV-radio-HPLC (Gilson, Middleton, USA) using an automated solid phase extraction controller (ASPEC Gilson) connected to a dilutor (Gilson), and a radio detector (Radiomatic 610TR, Packard, USA) coupled in series with a UV detector. The separation was performed on an Xbridge Prep BEH130 C18 (peptide separation technology) 250 mm×10 mm, 5 μm with a 10×10 mm C18 security guard from the same supplier. The HPLC system was operated at a flow rate of 6 ml/min. The mobile phase consisted of 0.1% TFA in MilliQ: 0.1% TFA in Acetonitrile. Gradient elution mode was used for the separation (Gradient: 0-5 min: 20-45%, 5-7 min: 45%, 7-10 min: 45-80%, 10-15 min: 80%). The outlet from the detector was connected to a switching valve on the arm of the ASPEC to enable automatic fraction collection. Five fractions were collected, and the radioactivity in the fractions was measured by a well-type scintillation counter. The radio-HPLC radiochromatograms were analysed for area under the curve for all peaks. The amount of intact radiolabeled peptide at each time point was calculated as percentage of the sum of all peaks on the radiochromatogram.

### In Vitro Cellular Assays for GIP, GLP-1 and Glucagon Receptor Efficacy

Agonism of compounds at the human glucose-dependent insulinotropic polypeptide (GIP), glucagon-like peptide-1 (GLP-1), or glucagon (GCG) receptors was determined by functional assays measuring cAMP response of recombinant PSC-HEK-293 cell lines stably expressing human GIP, GLP-1 or glucagon receptors, respectively.

### 384-Well Format

The cells were grown in a T-175 culture flask placed at 37° C. to near confluence in medium (DMEM/10% FBS) and collected in 2 ml vials in cell culture medium containing 10% DMSO in concentration of 10-50 million cells/ml. Each vial contained 1.8 ml cells. The vials were slowly frozen to −80° C. in isopropanol, and then transferred in liquid nitrogen for storage.

Prior to their use, frozen cells were thawed quickly at 37° C. and washed (5 min at 900 rpm) with 20 ml cell buffer (lx HBSS; 20 mM HEPES, with 0.1% BSA). Cells were resuspended in assay buffer (cell buffer plus 2 mM IBMX) and adjusted to a cell density of 1 million cells/ml.

For measurement of cAMP generation, 5 μl cells (final 5000 cells/well) and 5 μl of test compound were added to a 384-well plate, followed by incubation for 30 min at room temperature.

The cAMP generated was determined using a kit from Cisbio Corp. based on HTRF (Homogenous Time Resolved Fluorescence). The cAMP assay was performed according to manufacturer's instructions (Cisbio).

After addition of HTRF reagents diluted in lysis buffer (kit components), the plates were incubated for 1 h, followed by measurement of the fluorescence ratio at 665/620 nm. In vitro potency of agonists was quantified by determining the concentrations that caused 50% activation of the maximal response (EC50).

### 96-Well Format

cAMP content of cells was determined using a kit from Cisbio Corp. (cat. no. 62AM4PEC) based on HTRF (Homogenous Time Resolved Fluorescence). For preparation, cells were split into T-175 culture flasks and grown overnight to near confluency in medium (DMEM/10% FBS). Medium was then removed and cells washed with PBS lacking calcium and magnesium, followed by proteinase treatment with accutase (Sigma-Aldrich cat. no. A6964). Detached cells were washed and resuspended in assay buffer (1×HBSS; 20 mM HEPES, 0.1% BSA, 2 mM IBMX) and cellular density determined. They were then diluted to 400000 cells/ml and 25 μl-aliquots dispensed into the wells of 96-well plates. For measurement, 25 μl of test compound in assay buffer was added to the wells, followed by incubation for 30 minutes at room temperature. After addition of HTRF reagents diluted in lysis buffer (kit components), the plates were incubated for 1 hr, followed by measurement of the fluorescence ratio at 665/620 nm. In vitro potency of agonists was quantified by determining the concentrations that caused 50% activation of maximal response (EC50).

### In Vitro Assays for Binding to the Human GIP Receptor

(1) Preparation of Membranes from HEK-293 Cells Over-Expressing the GIPR

HEK-293 cells recombinantly over-expressing GIPR were grown to 50% confluency, washed with warm 1×PBS (Gibco) and detached in HEPES/EDTA-buffer (100 mM HEPES pH 7.5, 5 mM EDTA). Cells were harvested by centrifugation at 4° C. and 3000×g and the pellets were stored at −80° C. until further processing.

After thawing on ice, pellets were resuspended in HEPES/EDTA-buffer and homogenized on ice for 1 min using Ultra-Turray T25. After subsequent sonification the cell debris was removed by centrifugation at 1000×g and 4° C. Supernatants were then ultra-centrifuged at 100000×g and 4° C. under vacuum for 30 min. Pellets were resuspended in HEPES/EDTA/NaCl-buffer (20 mM HEPES, 1 mM EDTA, 150 mM NaCl; add 1 Complete Mini Protease inhibitor cocktail to 10 ml buffer) and protein content was determined via BCA-Protein assay.

### (2) Measurement of Binding Activity of Test Compounds to Human GIPR

For the measurement of the binding activity to GIPR, [¹²⁵I]GIP (PerkinElmer), in a final concentration of 100 pM and a test compound in 10 concentrations were mixed with PVT-WGA SPA beads (0.125 mg/well; Perkin-Elmer) coated with HEK-293 cell membranes (1 μg/well of protein) expressing the GIPR in assay buffer [50 mM HEPES (pH 7.4, WAKO), 5 mM EGTA (WAKO), 5 mM MgCl₂ (WAKO), and 0.005% Tween 20 (BioRad)] and incubated at room temperature for 2 h. Specific binding was calculated as the difference between the amount of [¹²⁵I]labeled hot ligand bound in the absence (total binding) and presence (nonspecific binding) of 1 μM unlabeled cold reference ligand, respectively.

### EXAMPLES

The invention is further illustrated by the following examples.

### Example 1: Synthesis of VS-DO3A building block ([4,10-Bis-carboxymethyl-7-(2-ethenesulfonyl-ethyl)-1,4,7,10-tetraaza-cyclododec-1-yl]-acetic acid)

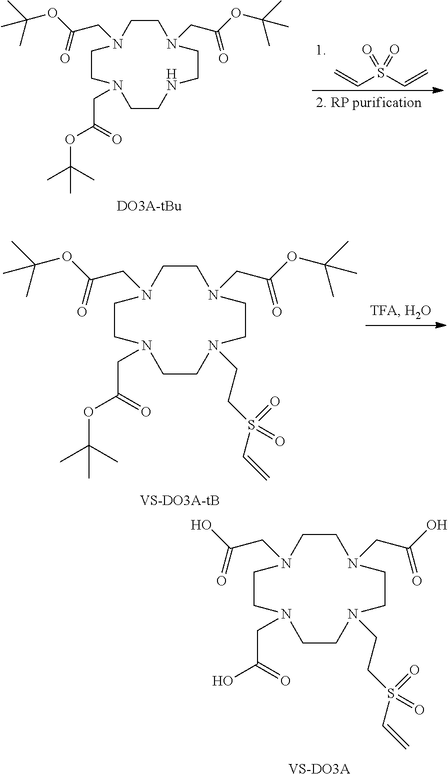

To a solution of DO3A-tBu (4,10-Bis-tert-butoxycarbonylmethyl-1,4,7,10-tetraaza-cyclododec-1-yl)-acetic acid tert-butyl ester (2.5 g) in DMF (10 mL) was at 0° C. added a solution of divinyl sulfone (5 mL) in DMF/water 1:1 (20 mL). The mixture was allowed to reach room temperature and was stirred for 2 h. The mixture was directly purified by RP chromatography to give VS-DO3A-tBu ([4,10-Bis-tertbutoxycarbonylmethyl-7-(2-ethenesulfonyl-ethyl)-1,4,7,10-tetraaza-cyclododec-1-yl]-acetic acid tert-butyl ester).

A solution of VS-DO3A-tBu in TFA/water 19:1 (75 mL) was stirred at room temperature for 1 day. TFA was carefully evaporated and the remaining solution was freeze-dried to give crude VS-DO3A ([4,10-Bis-carboxymethyl-7-(2-ethene sulfonyl-ethyl)-1,4,7,10-tetraaza-cyclododec-1-yl]acetic acid) which was directly used without further purification.

### Example 2: Synthesis of SEQ ID NO: 3

The solid phase synthesis as described in Methods was carried out on a 0.2 mmol scale on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.43 mmol/g. The Fmoc-synthesis strategy was applied with HBTU/DIPEA-activation. In position 1 Fmoc-Tyr-OH was used in the solid phase synthesis protocol. The peptide was cleaved from the resin with King's cocktail (D. S. King, C. G. Fields, G. B. Fields, Int. J. Peptide Protein Res. 36, 1990, 255-266). The crude product was purified via preparative HPLC on a Waters column (Sunfire, Prep C18) using an acetonitrile/water (with 0.1% TFA) gradient. The molecular mass of the purified peptide intermediate was confirmed by LC-MS. 84 mg of this peptide intermediate was dissolved in 12 mL H₂O and 2 mL ACN. 166 μL triethylamine was added to adjust the pH to pH11.3. The solution was cooled to 0° C. and a solution of DOTA-NHS ester (27 mg, 1.5 eq.; CAS-Nr. 170908-81-3) in 3 mL of 0.1% TFA in ACN was added dropwise over 5 minutes. pH control showed a pH of 10.3. The reaction mixture was stirred for 1 h at 0° C. Then additional DOTA-NHS ester (18 mg, 1 eq.) was added. Stirring was continued for another 1.5 h. The reaction was stopped by addition of aqueous AcOH until pH4 was reached. The solvent was removed by lyophilization. The crude product was purified via preparative HPLC on a Waters column (X-Select CSH, Prep C18) using an acetonitrile/water (with 0.1% formic acid) gradient. Finally, the molecular mass of the purified peptide was confirmed by LC-MS.

### Example 3: Synthesis of SEQ ID NO: 4

21.59 mg SEQ ID NO:3 was suspended in 18 mL pH4.6 acetate buffer. 6.13 mg Gallium(III) sulfate hydrate was added and the reaction mixture was heated to 80° C. for 15 minutes. After cooling to RT, the mixture was diluted with H₂O/ACN. pH was adjusted to pH7.4 by careful addition of aqueous NaOH. Solvents were then removed by lyophilization. The crude product was purified via preparative HPLC on a Waters column (X-Select CSH, Prep C18) using an acetonitrile/water (with 0.1% TFA) gradient.

Finally, the molecular mass of the purified peptide was confirmed by LC-MS.

### Example 4: Synthesis of SEQ ID NO: 5

The solid phase synthesis as described in Methods was carried out on a 0.1 mmol scale on Novabiochem Rink-Amide resin (4-(2′,4′-Dimethoxyphenyl-Fmoc-aminomethyl)-phenoxyacetamido-norleucylaminomethyl resin), 100-200 mesh, loading of 0.35 mmol/g. The Fmoc-synthesis strategy was applied with HBTU/DIPEA-activation. In position 1 Fmoc-Tyr-OH was used in the solid phase synthesis protocol. The peptide was cleaved from the resin with King's cocktail (D. S. King, C. G. Fields, G. B. Fields, Int. J. Peptide Protein Res. 36, 1990, 255-266). The crude product was purified via preparative HPLC on a Waters column (X-Select CSH, Prep C18) using an acetonitrile/water (with 0.1% TFA) gradient. The molecular mass of the purified peptide intermediate was confirmed by LC-MS.

36.7 mg of this peptide intermediate was dissolved in 5 mL pH9.2 borate buffer. Some drops ACN were added to give a slightly turbid reaction mixture. 12.1 mg (2.5 eq.) VS-DO3A building block ([4,10-Bis-carboxymethyl-7-(2-ethenesulfonyl-ethyl)-1,4,7,10-tetraaza-cyclododec-1-yl]acetic acid) (see example 1) were added. The pH was readjusted from pH5 to pH7.1 by dropwise addition of pH10 buffer. The turbid reaction mixture was stirred overnight at RT. Then 20 mL water was added and the solvent removed by lyophilization. The crude product was purified via preparative HPLC on a Waters column (X-Select CSH, Prep C18) using an acetonitrile/water (with 0.1% TFA) gradient.

Finally, the molecular mass of the purified peptide was confirmed by LC-MS.

In an analogous way, the peptide SEQ ID NO: 6 can be synthesized.

TABLE 4
List of synthesized peptides and comparison
of calculated vs. found molecular weight

| | | | Monoisotopic | Retention | |
|---|---|---|---|---|---|
| SEQ | calc. Mass | found | or average | time | |
| ID NO | monoisotopic | mass | mass | [min] | Method |
| 3 | 3920.00 | 3920.01 | monoisotopic | 11.1 | B |
| 4 | 3985.90 | 3985.91 | monoisotopic | 11.2 | B |
| 5 | 3971.94 | 3971.94 | monoisotopic | 10.4 | B |

### Example 5: In Vitro Data on Potency GIP, GLP-1 and Glucagon Receptors

Potencies of peptidic compounds at the GIP, GLP-1 and glucagon receptors were determined by exposing cells expressing human GIP (hGIPR), human GLP-1 receptor (hGLP-1R), and glucagon receptor (hGCGR) to the listed compounds at increasing concentrations and measuring the formed cAMP as described in Methods.

The results for GIP(1-30) analogs with agonistic activity at the human GIP receptor (hGIPR) are shown in Table 5.

TABLE 5
Potency expressed as EC50 values of GIP(1-30) analogs
at GIP, GLP-1 and Glucagon receptors (indicated in pM)

| | EC50 | EC50 | EC50 | |
|---|---|---|---|---|
| SEQ | hGIP R | hGLP-1 R | hGCGR | Well |
| ID NO | [pM] | [pM] | [pM] | format |
| 1 | 0.49 | >10000 | >10000 | 96 |
| 1 | 0.19 | >10000 | >10000 | 384 |
| 2 | 0.40 | >10000 | >10000 | 96 |
| 3 | 1.13 | >10000 | >10000 | 96 |
| 4 | 1.09 | >10000 | >10000 | 96 |
| 5 | 0.22 | >10000 | >10000 | 384 |
| hGLP-1 | >10000 | 1.26 | >10000 | 96 |
| hGLP-1 | >10000 | 0.70 | >10000 | 384 |
| hGlucagon | >10000 | 42.3 | 0.49 | 96 |
| hGlucagon | >10000 | 16.0 | 0.19 | 384 |

As shown by the higher EC50 values in Table 5, the inventive peptides represented by SEQ ID NOs: 3, 4 and 5 had a lower potency, i.e. ability to activate the human GIP receptor, compared to native GIP (SEQ ID NO: 1) or the GIP(1-30) truncated peptide.

### Example 6: In Vitro Affinity Data for the Human GIP Receptor (Binding Assay)

Affinity of peptidic compounds to the human GIP receptor were determined as described in Methods.

The results are shown in Table 6.

TABLE 6
Affinity expressed as IC50 values at human GIP receptor (in nM)

| SEQ | IC50 hGIPR |
|---|---|
| ID NO | [nM] |
| 1 | 3.13 |
| 2 | 1.3 |
| 4 | 0.25 |
| 5 | 0.22 |
| hGLP-1 | >10000 |

As shown by the lower IC50 values in Table 6, the inventive peptides had a binding affinity that was improved in relation to GIP(1-30) and native GIP by approximately 5 and 12 times, respectively,

### Example 7: Solubility

Peptide samples were prepared in the respective solubility buffer system and solubility was assessed as described in Methods. The results are given in Table 7.

TABLE 7
Solubility of peptides

| SEQ | Solubility buffer | Soluble amount |
|---|---|---|
| ID NO | system | [mg/ml] |
| 3 | A | ≥0.5 |
| 3 | B | <0.5 |
| 3 | C | ≥0.5 |
| 3 | D | ≥0.5 |
| 3 | E | ≥0.5 |
| 3 | F | ≥0.5 |

### Example 8: Chemical Stability

Chemical stability of peptidic compounds was assessed as described in Methods. The results are given in Table 8.

TABLE 8
Chemical stability of peptides

| SEQ | | |
|---|---|---|
| ID NO | Temperature | % Purity after 7 days |
| 3 | 4° C. | >97 |
| | 40° C. | >97 |

### Example 9: Plasma Stability

Plasma stability of SEQ ID NO: 4 was assessed as described in Methods.

The peptide demonstrated stability in plasma from NHP, with in excess of 92% of the radioactive signal arising from intact peptide after 90 minutes incubation.

TABLE 9
Stability of SEQ ID NO: 4 in NHP plasma.

| Time (min) | Intact peptide (%) |
|---|---|
| 0 | 99.5 |
| 15 | 98.65 |
| 45 | 96.31 |
| 90 | 92.98 |

The demonstrated stability of the inventive peptide in plasma is considerably higher than reported previously for GIP(1-42) (Deacon C F, Nauck M A, Meier J, Hücking K, Holst J J. J Clin Endocrinol Metab. 2000; 85(10):3575-3581).

The invention is further characterized by the following items:

Item 1. Compounds of the formula I

(I)
Tyr-Aib-Glu-Gly-Thr-Phe-Ile-Ser-Asp-Leu-Ser-Ile-
Ala-Leu-Asp-Arg-Ile-His-Gln-Glu-Glu-Phe-Ile-X24-
Trp-Leu-Leu-Ala-Gly-Gly-X31-R¹

X24 represents an amino acid selected from Glu or Gln,
X31 represents an amino acid selected from Cys(VS-DO3A), Cys(VS-NO2A),
Cys(mal-DOTA), Cys(mal-NOTA), Cys(mal-NODAGA), Lys(DOTA),
Lys(NOTA), Lys(PEG-DOTA) and Lys(VS-DO3A),
   wherein DOTA, NOTA, DO3A, NO2A or NODAGA may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺,
   R¹ represents OH or NH₂
or a salt or a solvate thereof.

Item 2. Compounds of item 1, which are capable of activating the human GIP receptor.

Item 3. Compounds of items 1 to 2, which are an agonist at the human GIP receptor.

Item 4. Compounds of items 1 to 3, which are capable of activating the human GIP receptor in an assay with whole cells expressing the human GIP receptor.

Item 5. Compounds of items 1 to 4, having an EC50 for hGIP receptor as determined by the method of Example 5 of 10 pM or less.

Item 6. Compounds of items 1 to 4, having an EC50 for hGIP receptor as determined by the method of Example 5 of 5 pM or less.

Item 7. Compounds of items 1 to 4, having an EC50 for hGIP receptor as determined by the method of Example 5 of 2 pM or less.

Item 8. Compounds of items 1 to 7, having a lower EC50 for hGIP receptor than at the human GLP-1 receptor.

Item 9. Compounds of any one of items 1 to 8, having an EC50 for hGLP-1 receptor as determined by the method of Example 5 of 100 pM or more.

Item 10. Compounds of any one of items 1 to 8, having an EC50 for hGLP-1 receptor as determined by the method of Example 5 of 1000 pM or more.

Item 11. Compounds of any one of items 1 to 8, having an EC50 for hGLP-1 receptor as determined by the method of Example 5 of 10000 pM or more.

Item 12. Compounds of items 1 to 11, having a lower EC50 for hGIP receptor than at the human Glucagon receptor.

Item 13. Compounds of any one of items 1 to 11, having an EC50 for hGlucagon receptor as determined by the method of Example 5 of 100 pM or more.

Item 14. Compounds of any one of items 1 to 11, having an EC50 for hGlucagon receptor as determined by the method of Example 5 of 1000 pM or more.

Item 15. Compounds of any one of items 1 to 11, having an EC50 for hGlucagon receptor as determined by the method of Example 5 of 10000 pM or more.

Item 16. Compounds of any one of items 1 to 15 binding to the hGIP receptor as determined using the method of Example 6 with an IC50 of 100 nM or less.

Item 17. Compounds of any one of items 1 to 15 binding to the hGIP receptor as determined using the method of Example 6 with an IC50 of 10 nM or less.

Item 18. Compounds of any one of items 1 to 15 binding to the hGIP receptor as determined using the method of Example 6 with an IC50 of 3.13 nM or less.

Item 19. Compounds of any one of items 1 to 15 binding to the hGIP receptor as determined using the method of Example 6 with an IC50 of 1 nM or less.

Item 20. Compounds of any one of items 1 to 19 having a solubility for metal ion loading procedure of at least 0.5 mg/ml.

Item 21. Compounds of any one of items 1 to 20 having a high chemical stability when stored in solution.

Item 22. Compounds of any one of items 1 to 20 having a high chemical stability that after 7 days at 40° C. in solution at pH 7.3 the relative purity loss is no more than 10%.

Item 23. Compounds of any one of items 1 to 20 having a high chemical stability that after 7 days at 40° C. in solution at pH 7.3 the relative purity loss is no more than 5%.

Item 24. Compounds of any one of items 1 to 20 having a high chemical stability that after 7 days at 40° C. in solution at pH 7.3 the relative purity loss is no more than 3%.

Item 25. Compounds of any one of items 1 to 24 which can be used for in vivo imaging of GIP receptor expressing cells in humans.

Item 26. Compounds of any one of items 1 to 24 which can be used for in vivo imaging of GIP receptor expressing cells in the human pancreas.

Item 27. Compounds of any one of items 1 to 26 having the formula (I), wherein
X31 represents an amino acid Cys(VS-DO3A) or Lys(DOTA),
wherein DOTA or DO3A may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺.

Item 28. Compounds of any one of items 1 to 27 having the formula (I), wherein
X24 is Glu.

Item 29. Compounds of any one of items 1 to 28 having the formula (I), wherein
X24 is Glu, and
R¹ is OH.

Item 30. Compounds of any one of items 1 to 28 having the formula (I), wherein
X24 is Glu, and
R¹ is NH₂.

Item 31. Compounds of any one of items 1 to 27 having the formula (I), wherein
X24 is Gln.

Item 32. Compounds of any one of items 1 to 27 having the formula (I), wherein
X24 is Gln, and
R¹ is OH.

Item 33. Compounds of any one of items 1 to 27 having the formula (I), wherein
X24 is Gln, and
R¹ is NH₂.

Item 34. Compounds of any one of items 1 to 27 having the formula (I), wherein
X24 is Gln,
X31 is C(VS-DO3A).

Item 35. Compounds of any one of items 1 to 27 having the formula (I), wherein
X24 is Glu,
X31 is K(DOTA).

Item 36. Compounds of SEQ ID NO: 3 and 5, as well as salts or solvates thereof.

Item 37. Compounds of SEQ ID NO: 4 and 6, as well as salts or solvates thereof.

Item 38. Compounds of any one of items 1 to 27 having the formula (I), wherein DOTA or DO3A is unloaded.

Item 39. Compounds of any one of items 1 to 27 having the formula (I), wherein
DOTA or DO3A is loaded with a metal ion selected from Gd³⁺, Ga³⁺, Cu²⁺, (Al—F)²⁺, Y³⁺, Tc³⁺, In³⁺, Lu³⁺ and Re³⁺.

Item 40. Compounds of item 39 having the formula (I), wherein
DOTA or DO3A is loaded with a metal ion Ga³⁺.

Item 41. Compounds of item 39 having the formula (I), wherein
DOTA or DO3A is loaded with a metal ion Gd³⁺.

Item 42 Compounds of item 39 having the formula (I), wherein
DOTA or DO3A is loaded with a metal radionucleotide ion (Cu-64)²⁺, (Ga-68)³⁺, (Al—F-18)²⁺, (Y-86)³⁺.

Item 43. Compounds of item 39 having the formula (I), wherein
DOTA or DO3A is loaded with one metal radionucleotide ion (Ga-67)³⁺, (Tc-99)³⁺, (In-111)³⁺.

Item 44. Compounds of item 39 having the formula (I), wherein
DOTA or DO3A is loaded with one metal radionucleotide ion selected from (Cu-67)²⁺, (Y-90)³⁺, (In-111)³⁺, (Lu-177)³⁺, (Re-186)³⁺ and (Re-188)³⁺.

Item 45. Use of Compounds of any one of items 1 to 36 having the formula (I),

wherein DOTA or DO3A is loaded with a metal radionucleotide ion selected from (Cu-67)²⁺, (Y-90)³⁺, (In-111)³⁺, (Lu-177)³⁺, (Re-186)³⁺ and (Re-188)³⁺, in Peptide receptor radionuclide therapy (PRRT).

Item 46. Use of Compounds of any one of items 1 to 36 having the formula (I),

wherein DOTA or DO3A is loaded with a metal radionucleotide ion selected from (Cu-67)²⁺, (Y-90)³⁺, (In-111)³⁺, (Lu-177)³⁺, (Re-186)³⁺ and (Re-188)³⁺ to treat neuroendocrine tumors (NETs) with elevated expression of GIP receptors.

Item 47. Use according to item 45 or 46 wherein the radionucleotide is (Lu-177)³⁺.

1. A peptidic compound having the formula (I):
(I)
Tyr-Aib-Glu-Gly-Thr-Phe-Ile-Ser-Asp-Leu-Ser-Ile-
Ala-Leu-Asp-Arg-Ile-His-Gln-Glu-Glu-Phe-Ile-X24-
Trp-Leu-Leu-Ala-Gly-Gly-X31-R¹
wherein
X24 represents an amino acid selected from Glu or Gln,
X31 represents an amino acid selected from Cys(VS-DO3A), Cys(VS-NO2A), Cys(mal-DOTA), Cys(mal-NOTA), Cys(mal-NODAGA), Lys(DOTA), Lys(NOTA), Lys(PEG-DOTA) and Lys(VS-DO3A),
wherein DOTA, NOTA, DO3A, NO2A or NODAGA may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺,
R¹ represents OH or NH₂
or a salt or a solvate thereof.

2. The compound of claim 1 having the formula (I), wherein
X31 represents an amino acid Cys(VS-DO3A) or Lys(DOTA),
wherein DOTA or DO3A may be unloaded or loaded with a metal ion selected from Gd³⁺, Ga³⁺, Cu²⁺, (Al—F)²⁺, Y³⁺, Tc³⁺, In³⁺, Lu³⁺ or Re³⁺.

3. The compound of claim 1, selected from the group consisting of compounds of SEQ ID NOs: 3 to 6 and salts or solvates thereof.

4. The compound of claim 3, selected from the group consisting of SEQ ID NOs: 3 and 5, as well as salts or solvates thereof.

5. The compound of claim 3, selected from the group consisting of SEQ ID NOs: 4 and 6, as well as salts or solvates thereof.

6. The compound of claim 1 having the formula (I), wherein
DOTA or DO3A is unloaded.

7. The compound of claim 1 having the formula (I), wherein
DOTA or DO3A is loaded with a metal ion selected from Gd³⁺, Ga³⁺, Cu²⁺, (Al—F)²⁺, Y³⁺, Tc³⁺, In³⁺, Lu³⁺ and Re³⁺.

8. The compound of claim 7 having the formula (I), wherein
DOTA or DO3A is loaded with one metal radionucleotide ion selected from (Cu-67)²⁺, (Y-90)³⁺, (In-111)³⁺, (Lu-177)³⁺, (Re-186)³⁺ and (Re-188)³⁺, (Cu-64)²⁺, (Ga-68)³⁺, (Al—F-18)²⁺, (Y-86)³⁺, (Ga-67)³⁺, (Tc-99)³⁺, (In-111)³⁺, Ga³⁺, Gd³⁺.

9. A compound according to claim 1 for use in medicine, particularly in human medicine.

10. A compound according to claim 1 having the formula (I), wherein DOTA or DO3A is loaded with a metal radionucleotide ion selected from (Cu-67)²⁺, (Y-90)³⁺, (In-111)³⁺, (Lu-177)³⁺, (Re-186)³⁺ and (Re-188)³⁺, for use in Peptide receptor radionuclide therapy (PRRT).

11. A compound according to claim 1 having the formula (I), wherein DOTA or DO3A is loaded with a metal radionucleotide ion selected from (Cu-67)²⁺, (Y-90)³⁺, (In-111)³⁺, (Lu-177)³⁺, (Re-186)³⁺ and (Re-188)³⁺ for use in treatment of neuroendocrine tumors (NETs) with elevated expression of GIP receptors.

12. A compound for use according to claim 10 wherein the radionucleotide is (Lu-177)³⁺.

13. The compound according to claim 8 for use in radiotherapy and/or PET imaging.

14. A compound for use according to claim 9, wherein said compound is present as an active agent in a pharmaceutical composition together with at least one pharmaceutically acceptable carrier.

15. A pharmaceutical composition comprising at least one compound according to claim 1 or a physiologically acceptable salt or solvent thereof, and a pharmaceutically acceptable carrier.
